(19) United States
(12) Patent Application Publication
(10) Pub. No.: US 2010/0168557 A1
(43) Pub. Date: Jul. 1, 2010

(54) MULTI-ELECTRODE ABLATION SENSING CATHETER AND SYSTEM

(76) Inventors: D. Curtis Deno, Andover, MN (US); Jeffrey A. Schweitzer, St. Paul, MN (US); John A. Hauck, Shoreview, MN (US)

Correspondence Address: SJM/AFD - DYKEMA;c/o CPA Global, P.O. Box 52050, Minneapolis, MN, 55402, US

(21) Appl. No.: 12/346,592

(22) Filed: Dec. 30, 2008

(51) Int. Cl. A61B 18/18; A61B 5/05; A61B 18/14; A61B 5/053

(52) U.S. Cl. 600/424; 606/33; 606/41; 600/547

(57) ABSTRACT

The invention is directed to a multi-electrode ablation sensing catheter and system suitable for medical procedures such as cardiac ablation. In one embodiment of the invention, a catheter is provided having an elongated catheter shaft and a catheter tip having two or more closely spaced electrodes mounted on the catheter tip, where the electrodes are coupled to a plurality of electronic circuitries and are used for electrogram sensing, impedance sensing, and location sensing and orientation. In another embodiment of the invention, a catheter system is provided having a catheter with an elongated catheter shaft and a catheter tip with two or more closely spaced electrodes mounted on the catheter tip, and an RF generator circuitry, an electrogram sensing circuitry, an impedance sensing circuitry, and a location sensing and orientation circuitry.

### BACKGROUND OF THE INVENTION

a. Field of the Invention

In general, the invention relates to ablation catheters. More particularly, the invention relates to a multi-electrode ablation sensing catheter and system.

b. Background Art

It is known that catheters are widely used to perform a variety of functions relating to therapeutic and diagnostic medical procedures involving tissues within a body. For example, catheters may be inserted within a vessel located near the surface of a body (e.g., in an artery or vein in the leg, neck, or arm) and maneuvered to a region of interest within the body to enable diagnosis and/or treatment of tissue without the need for more invasive procedures. For example, catheters may be inserted into a body during ablation and mapping procedures performed on tissue within a body. Tissue ablation may be accomplished using a catheter to apply localized radiofrequency (RF) energy to a selected location within the body to create thermal tissue necrosis. Typically, the ablation catheter is inserted into a vessel in the body, sometimes with the aid of a pull wire or introducer, and threaded through the vessel until a distal tip of the ablation catheter reaches the desired location for the procedure. The ablation catheters commonly used to perform these ablation procedures produce lesions and electrically isolate or render the tissue non-contractile at various points in the cardiac tissue by physical contact of the cardiac tissue with an electrode of the ablation catheter and application of energy, such as RF energy. By way of further example, another procedure, mapping, may employ a catheter with sensing electrodes to monitor various forms of electrical activity in the body. Mapping can locate abnormal areas in the heart's electrical system.

Several challenges with known catheters, such as those used for ablation procedures, include ensuring improved contact between the catheter electrode(s) and the tissue to enable adequate electrogram sensing and application of RF ablation energy, ensuring adequate monitoring of ablation lesion size and location, and ensuring adequate catheter tip orientation and position visualization. Tissue contact is important for obtaining proper sensing of cardiac electrogram (EGM) signals. Without improved contact, signal amplitudes may be too small to reliably characterize nearby myocardium. Fractionated electrogram signals consist of small, high frequency, spike-like deflections which may be difficult to distinguish from electrical noise or more distant cardiac electrical events. Moreover, tissue contact is also an aspect of catheter ablation for arrhythmias. The destruction of pathologic cardiac tissue involves the delivery of energy, or removal of energy if cryoablation is performed, to a small controlled region. RF current spreads out from the ablation electrode, usually located at or about the catheter's tip. Heat damage occurs in the region where RF current density is high, before it dissipates through adjacent structures and returns to a cutaneous return electrode.

Known catheters, such as those used for ablation procedures, may include RF ablation catheters having large distal tips with several large, spaced electrodes affixed to the tip. However, due to the size of the electrodes and the large spacing between the electrodes, such catheter tip configurations may not provide improved tissue contact, adequate monitoring of ablation lesion size and location, and/or adequate catheter tip orientation and position visualization.

In addition, known catheters, such as those used for ablation procedures, typically rely on delivered power, tip temperature, and dwell time, all of which are indirect indices, to monitor ablation lesion location and size, as well as orientation, location, and contact of the ablation catheter's tip. However, such indirect indices can prove to be unreliable or inaccurate. Moreover, known ablation catheters may use impedance to reflect tissue contact and ablation induced tissue change. However, such changes may not be adequately robust and may serve more as an alert to the presence of coagulated blood covering the ablation electrode or gross contact issues that limit ablation efficacy. In addition, lesion size has not been well correlated to impedance. The poor reliability of impedance challenges to lesion size and contact may derive from impedance measurements made with excessively large electrodes on known ablation catheter tips.

Accordingly, there remains a need for a multi-electrode ablation sensing catheter and system that can be used for medical procedures including improved ablation therapies or treatment.

### BRIEF SUMMARY OF THE INVENTION

It is desirable to provide a multi-electrode ablation sensing catheter and system that can be used for medical procedures such as ablation that has a novel ablation catheter tip comprising multiple, closely-spaced, small electrodes, operating in parallel for ablation current delivery. The multi-electrode ablation sensing catheter and system of the invention provides for improved electrogram signal sensing by ensuring improved tissue contact and by using smaller sized electrodes that selectively sense nearby electrogram signals and that do not spatially and temporally integrate the electrogram signals farther away. The multi-electrode ablation sensing catheter and system of the invention further ensures improved contact between the catheter electrodes and the tissue for improved electrogram sensing and application of RF ablation energy, improved monitoring of ablation lesion size and location, and improved catheter tip orientation and position visualization. The multi-electrode ablation sensing catheter of the invention provides enhanced information regarding the location and orientation of the tip electrodes, uses impedance to determine the quality of the tip electrode contact, provides enhanced electrogram resolution, and provides enhanced pacing to minimize impact on electrogram signals and sensing. The multi-electrode ablation sensing catheter of the invention may also be assembled via a pre-manufactured piece part construction or assembly which can be less time consuming and tedious than conventional manual construction of known ablation catheters.

In one of the embodiments of the invention, a catheter is provided comprising: an elongated catheter shaft having a proximal end, a distal end, and a lumen therethrough; a catheter tip at the distal end of the catheter shaft having two or more closely spaced electrically active elements mounted on the catheter tip, wherein the electrically active elements are coupled to a plurality of electronic circuitries used for electrogram sensing, impedance sensing, and location sensing and orientation.

In another embodiment of the invention, a catheter system is provided, the catheter system comprising: a catheter having an elongated catheter shaft with a proximal end, a distal end, and a lumen therethrough, and having a catheter tip at the distal end of the catheter shaft with a plurality of closely spaced electrically active elements mounted on the catheter tip; RF generator circuitry for applying RF energy across the electrically active elements to a distant return electrically active element; electrogram sensing circuitry for sensing electrogram signals from the electrically active elements; impedance sensing circuitry for applying impedance current across the electrically active elements to the distant return electrically active element; and, location sensing and orientation circuitry for determining the catheter tip location and orientation. The catheter system may further comprise pacing output circuitry for minimizing interference with impedance sensing and location sensing and orientation.

The electrically active elements of both the catheter and catheter system are preferably electrodes spaced apart from each other a distance of 0.1 millimeter to 0.3 millimeter and positioned circumferentially around the catheter tip. In another embodiment of the invention, the electrically active elements may each have one or more protrusions on an outer surface.

In another embodiment of the invention, an ablation sensing catheter system is provided, the catheter system comprising: a catheter having an elongated catheter shaft with a proximal end, a distal end, and a lumen therethrough, and having a catheter tip at the distal end of the catheter shaft with a plurality of closely spaced electrodes mounted on the catheter tip; RF generator circuitry for applying RF energy across the electrodes to a distant return electrode; electrogram sensing circuitry for sensing electrogram signals from the electrodes; impedance sensing circuitry for applying impedance current across the electrodes to the distant return electrode; and, location sensing and orientation circuitry for determining the catheter tip location and orientation. The catheter system may further comprise pacing output circuitry for minimizing interference with impedance sensing and location sensing and orientation. The electrodes are preferably spaced apart from each other a distance of 0.1 millimeter to 0.3 millimeter and positioned circumferentially around the catheter tip.

The foregoing and other aspects, features, details, utilities, and advantages of the invention will be apparent from reading the following description and claims, and from reviewing the accompanying drawings.

### BRIEF DESCRIPTION OF THE DRAWINGS

FIG. 1 is a fragmentary view of one of the embodiments of the multi-electrode ablation sensing catheter of the invention.

FIG. 2 is a fragmentary view of one of the embodiments of the distal end of the multi-electrode ablation sensing catheter of the invention.

FIG. 3 is a fragmentary view of the multi-electrode portion of the tip of FIG. 2.

FIG. 4 is a fragmentary view of another embodiment of the multi-electrode ablation sensing catheter tip of the invention.

FIG. 5 is a fragmentary view of yet another embodiment of the multi-electrode ablation sensing catheter tip of the invention.

FIG. 6 is a fragmentary view in partial cross-section of a piece part construction of one of the embodiments of the multi-electrode ablation sensing catheter of the invention.

FIG. 7 is a fragmentary view in partial cross-section of the catheter of FIG. 6 with a press fit shaft.

FIG. 8 is a diagram of the electrically active elements of the catheter tip of FIG. 2 at a first angle of incidence contact position.

FIG. 9 is a diagram of the electrically active elements of the catheter tip of FIG. 2 at a second angle of incidence contact position.

FIG. 10 is a diagram of the electrically active elements of the catheter tip of FIG. 2 at a third angle of incidence contact position.

FIG. 11 is a diagram of the catheter tip of FIG. 2 implementing an RF generator.

FIG. 12 is a block diagram of the RF generator circuitry.

FIG. 13 is a block diagram of the electrogram sensing circuitry.

FIG. 14 is a block diagram of the impedance sensing circuitry.

FIG. 15 is a block diagram of the location sensing and orientation circuitry.

FIG. 16 is a block diagram of the pacing output circuitry.

### DETAILED DESCRIPTION OF THE INVENTION

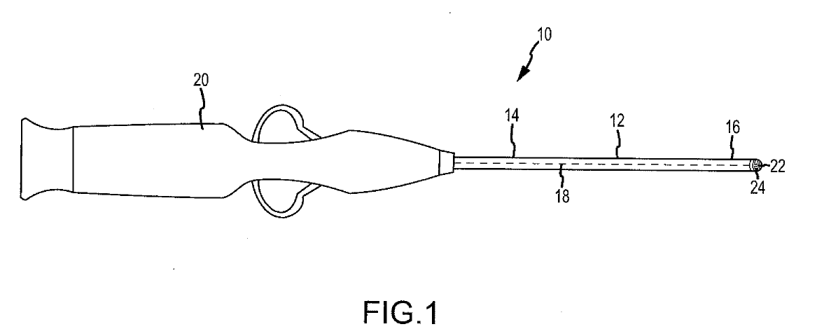

A catheter and catheter system provided in accordance with the teachings of the invention may be used in various therapeutic and/or diagnostic applications, such as the performance of a cardiac ablation procedure and other similar applications/procedures. Accordingly, one of ordinary skill in the art will recognize and appreciate that the inventive catheter and catheter system can be used in any number of therapeutic and/or diagnostic applications. The catheter and catheter system of the invention may be used for, among other things, ablation procedures on a human heart. Referring now to the figures, FIG. 1 is a fragmentary view of one of the embodiments of a multi-electrode ablation sensing catheter 10 of the invention. The catheter may be an RF ablation or a sensing catheter. The catheter 10 comprises an elongated catheter shaft 12 with a proximal end 14 and a distal end 16 and a lumen 18 therethrough. Additionally, the catheter shaft may include one or more additional lumens of various lengths. A handle 20 may be connected to the proximal end of the catheter and adapted for connection to the catheter. The handle 20 may further be adapted for connection to one or more actuation elements (not shown) so that a user of the catheter may selectively manipulate the distal end of the catheter assembly to deflect in one or more directions (e.g., up, down, left, and right). The handle 20 may be operative to effect movement (i.e., deflection) of the distal end of the catheter. The catheter shaft may, for example, be constructed of a flexible polymeric material, such as polyurethane, nylon, polyethylene, various types of plastic materials, such as PEBAX, or other suitable polymeric materials. (PEBAX is a registered trademark of Arkema France of France.) In an embodiment, the length of the catheter shaft may be from about 75 cm (centimeters) to about 150 cm.

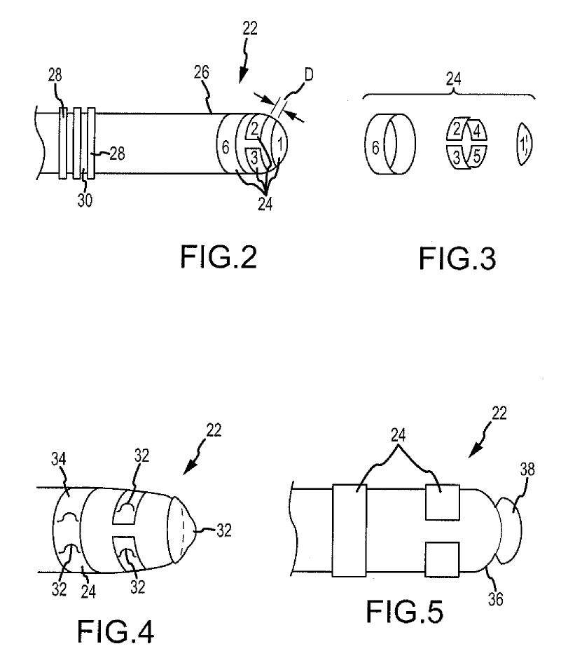

As shown in FIG. 1, the catheter further comprises a catheter tip 22 at the distal end 16 of the catheter shaft 12. The catheter tip 22 has two or more small, closely spaced electrically active elements 24 mounted on the catheter tip 22. FIG. 2 is a fragmentary view of one of the embodiments of the distal end of the multi-electrode ablation sensing catheter. The electrically active elements may comprise electrodes such as ablation electrodes, electrogram sensing electrodes, contact sensing electrodes, pacing electrodes, location electrodes, or sensors such as tissue impedance sensors or electrical sensors, or other suitable electrically active elements As shown in FIGS. 2 and 3, the electrically active elements 24 may be in the form of a plurality of smaller electrodes 1, 2, 3, 4, 5, 6 disposed on and spaced along an external surface 26 of the catheter tip 22. FIG. 3 is a fragmentary view of the multiple electrodes 1, 2, 3, 4, 5, 6 of the catheter tip of FIG. 2. In embodiments, the electrically active elements are small electrodes spaced apart from each other at a distance (D) of 0.1 millimeter to 0.3 millimeter. The spacing is preferably small enough such that when the electrodes are used in parallel, the electrode edge current density is increased only a small amount compared to the natural variation of current density which results from electrode contact with tissue. In this manner the edge effects are not as pronounced or as likely to cause additional coagulum. While some spacing is preferred between the multiple electrodes, this spacing is much smaller than the inter-electrode spacing between electrodes on conventional ablation catheters. The use of smaller spacing between the multiple electrodes helps to prevent coagulation. In an embodiment, the electrically active elements are positioned circumferentially around the catheter tip. However, the electrically active elements may also be positioned in other suitable configurations on the catheter tip. The catheter tip includes at least two electrically active elements mounted on the tip. For example, in the illustrated embodiment, the catheter tip has six electrically active elements mounted on the tip. However, the catheter tip may also have between two and five electrically active elements or electrodes or more than six electrically active elements or electrodes. The small electrically active elements may function as a single electrically active electrode or unit. The catheter of the invention has multiple small electrodes that provide improved electrogram resolution. These electrodes may be used for location sensing and orientation but may also be used for electrogram sensing. The macroscopic surface area of these electrodes may be on the order of 0.5 mm² (square millimeters) to 1.5 mm². These electrodes preferably have surface microtexturing or sufficient macroscopic size such that they do not exceed 2 k-ohms (kilohms) to 10 k-ohms Thevenin impedance for electrogram and location sensing frequencies. In this manner these electrodes will not predispose to noisy or artifact electrogram or location sensing signals. The impedance sensing circuitry, as discussed below, typically uses additional electrodes on the body surface. As shown in FIG. 2, the catheter shaft 12 may further include one or more additional ring electrodes 28 mounted circumferentially on an external surface 30 of the catheter shaft 12.

The catheter tip may also include a lumen (not shown) extending through at least a portion of the catheter tip in communication with the lumen or lumens of the catheter shaft. The catheter tip may, without limitation, be constructed of a material such as a polymeric material, a ceramic material, or another suitable material. The electrically active elements or electrodes may, without limitation, be constructed of a material such as platinum, platinum iridium, stainless steel, stainless steel alloys, gold, or another suitable material. In addition, the surfaces of the electrically active elements or electrodes may have surface coatings such as titanium nitride, iridium oxide, platinum black, or another suitable surface coating. Such surface coatings may be used to change a property or properties of the electrically active elements, such as, for example, the impedance properties or electrical properties.

FIG. 4 is a fragmentary view of another embodiment of the multi-electrode ablation sensing catheter tip 22 of the invention. In this embodiment one or more electrically active elements 24 have one or more protrusions or bumps 32 on an outer surface 34. Such a configuration with the electrodes protruding slightly from the catheter may enhance the concentration of contact force and local deformation. Wideband filtered ST segment changes of electrogram signals are a function of contact force and thus provide contact information and help to recognize early after depolarizations (EADs) and delayed after depolarizations (DADs). EADs and DADs are believed to contribute to fractionation and late potentials and provide ablation target information.

FIG. 5 is a fragmentary view of yet another embodiment of the multi-electrode ablation sensing catheter tip 22 of the invention having electrically active elements 24. In this embodiment a most distal portion 36 of the catheter tip 22 has an electrically active element in the form of a curved protrusion 38 mounted on the distal portion 36.

The electrically active elements are coupled to a plurality of electronic circuitries used for electrogram sensing, impedance sensing, and location sensing and orientation. The electronic circuitries preferably comprise an electrogram sensing circuitry, an impedance sensing circuitry, a location sensing and orientation circuitry, and an RF generator circuitry, all discussed in detail below. Optionally, the electronic circuitries may further comprise a pacing output circuitry, also discussed in detail below.

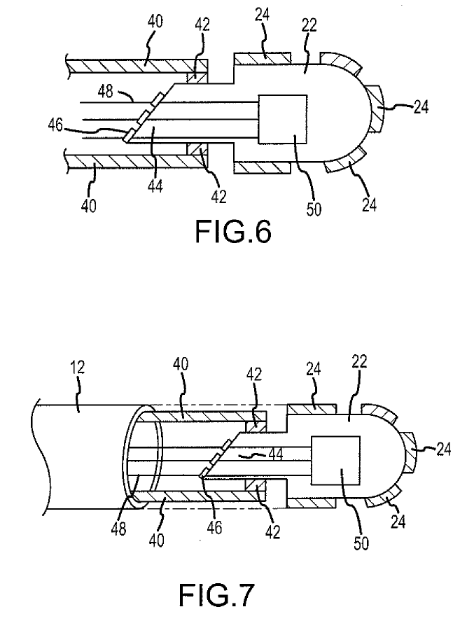

The catheter of the invention is preferably designed for manufacturability. The catheter may be assembled in a piece part assembly by mating the catheter shaft which is pre-manufactured with the catheter tip which is pre-manufactured. FIG. 6 is a fragmentary view in partial cross-section of the piece part construction or assembly of one of the embodiments of the multi-electrode ablation sensing catheter of the invention. FIG. 6 shows the catheter tip 22 having the electrically active elements 24, where the catheter tip 22 is coupled to pull wires 40 at weld 42, and where a proximal end 44 of the catheter tip 22 is further coupled to one or more wire bonding pads 46 which are attached to one or more electrode wires 48. The catheter tip may also include one or more integrated temperature sensors 50, liquid cooling elements (not shown) or other suitable components. The catheter may include one, two, or more pull wires. Conventional multi-electrode ablation catheters are typically assembled manually by putting individual electrodes on the catheter tip. Such assembly can be time consuming and tedious. The multi-electrode ablation sensing catheter of the invention may be pre-manufactured, such that the assembly process mates the pre-manufactured catheter shaft to the pre-manufactured catheter tip with the electrodes already formed on the tip. The electrodes may be assembled with the catheter tip using any number of known processes. For instance, the electrodes may be formed on the catheter tip using a reflow process. In such a process, the electrodes may be placed at the desired locations on the catheter tip, and then the catheter tip may be exposed to a heating process in which the electrodes and the catheter become affixed or bonded together. In an alternative process, the electrodes may be potted or cast into position and surrounded by polymer casting material.

FIG. 7 is a fragmentary view in partial cross-section of the catheter tip 22 of FIG. 6 with the catheter shaft 12 in the form of a press fit shaft. The catheter tip 22 may be press fitted and glued to the catheter shaft 12. In addition to the multiple electrodes and provision for a secure fit to the catheter shaft, the ablation catheter may be constructed to employ a pre-manufactured piece part integrated into the catheter shaft optionally with the convenient wire bonding pads 46, one or more electrode wires 48, pull wires 40 for deflection control, one or more integrated temperature sensors 50, liquid cooling elements (not shown) or other suitable components With this construction, the relative electrode spacing may be closely controlled obviating the need for individual calibration. The electrically active elements may be activated by electrical energy supplied through electrode wires 48 or additional electrode wires to the electrically active elements. It should be noted that while the embodiments described herein include components that may be primarily used for therapeutic and diagnostic applications, components for various other medical applications using such catheters may also be disposed within the catheter. In addition to the piece part assembly or construction, the catheter tip and catheter shaft may be formed using any number of different manufacturing processes known in the art including, without limitation, extrusion processes.

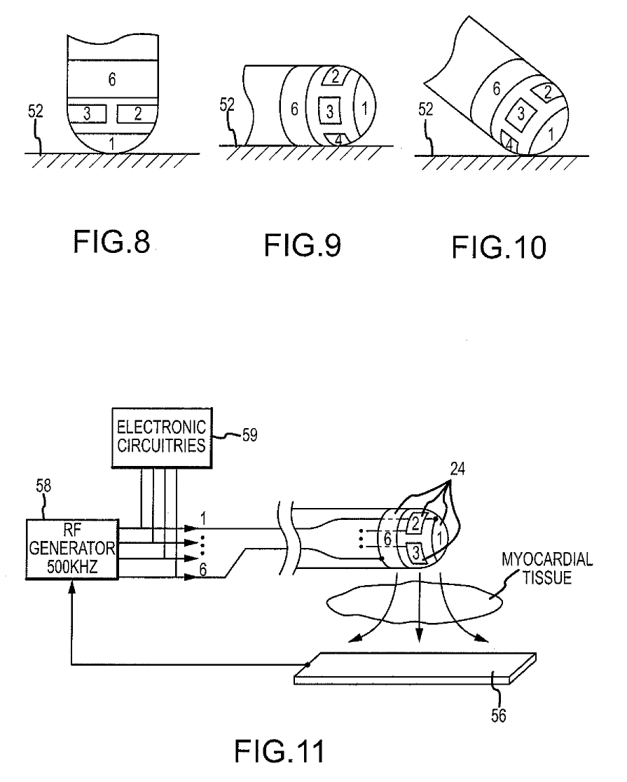

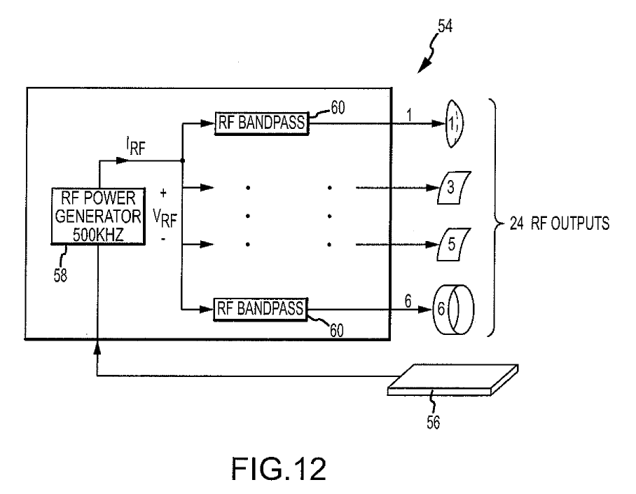

FIGS. 8-10 show diagrams of angles of incidence sensors accomplished by using the individual RF voltages and current sources as shown in FIG. 12 and discussed below. FIG. 8 is a diagram of the electrically active elements of the catheter tip of FIG. 2 at a first angle of incidence contact position where the angle of incidence is 90 degrees. When the catheter makes contact with just its tip, nearly perpendicular to a tissue surface 52, the most distal effective electrode (1) is most sensitive to sensing contact by an RF impedance rise. FIG. 9 is a diagram of the electrically active elements of the catheter tip of FIG. 2 at a second angle of incidence contact position where the angle of incidence is 0 degrees. When the catheter tip is in contact and fully longitudinally in contact with the tissue surface 52, the more proximal effective electrodes (4 and 6) are the most sensitive to sensing contact. FIG. 10 is a diagram of the electrically active elements of the catheter tip of FIG. 2 at a third angle of incidence contact position where the angle of incidence is 45 degrees. When the catheter tip is oriented at intermediate angles of incidence, contact with the tissue surface 52 is made with both effective electrodes (1 and 4). Other suitable angles of incidence for contacting the catheter tip to the tissue surface may also be used. A contact sensing system and method for assessing coupling between an electrode and tissue is disclosed in U.S. patent application Ser. No. 12/253,637, filed Oct. 17, 2008, which is a continuation-in-part of U.S. patent application Ser. No. 12/095,688, filed May 30, 2008, the entire disclosures of which are incorporated herein by reference. The configuration of the catheter of the invention is suited for an angle of incidence or angle of attack by virtue of the different combinations of electrodes that end up contacting the tissue surface, and the information obtained may be utilized as an angle of contact piece of information. Thus, at nearly any angle of contact, the catheter will have one or more electrodes contacting the tissue surface. Such contact can be measured and the angle of contact can be determined by the degree to which a particular combination of electrodes senses contact.

In another embodiment of the invention, there is provided a catheter system. The catheter system comprises a catheter having an elongated catheter shaft with a proximal end and a distal end and a lumen therethrough, and having a catheter tip at the distal end of the catheter shaft with a plurality of closely spaced electrically active elements mounted on the catheter tip, as discussed above and shown in FIGS. 1-2. Preferably, the electrically active elements are electrodes spaced apart from each other a small distance of 0.1 millimeter to 0.3 millimeter. Preferably, the electrically active elements are positioned circumferentially around the catheter tip. The catheter system further comprises RF generator circuitry 54 (see FIG. 12) for applying RF energy across two or more of the electrically active elements to a distant return electrically active element or electrode 56 (see FIG. 12). Preferably, the energy passed onto the electrodes is RF (radiofrequency) energy. For ablation procedures, the frequency may be 500 kHz (kilohertz). However, different frequencies may be used for different applications. The two or more electrodes at the distal end of the ablation catheter form a single effective electrode with the aid of the RF energy or generator circuitry. Effectively parallel RF current application is achieved by multiple active circuits to apply the same RF waveform to each electrode, such that they are effectively isopotential during the application.

FIG. 11 is a diagram of the tip of FIG. 2 implementing RF ablation. The one or more electrically active elements 24, in the form of electrodes 1-6, together form a single composite ablation electrode for the purpose of treating adjacent myocardial tissue. An RF generator 58, preferably having a frequency of 500 kHz (kilohertz) or another suitable frequency, provides power to all six electrodes or electrically active elements and collects the power at the distant return electrode or electrically active element 56. Individual electrode element connections are provided to other electronic circuitries 59 for electrogram sensing, location sensing and orientation, pacing, or impedance. This is achieved without interference.

FIG. 12 is a block diagram of the RF generator circuitry 54. The RF generator 58 generates voltage (V_{RF}) and current (I_{RF}) to RF band pass filter elements 60, which in turn, flow to electrically active elements or electrodes 24 as RF output. RF band pass filter elements 60 are tuned to pass the 500 kHz (kilohertz) RF power signal and filter out other frequencies, such as electrogram frequencies (0.1-500 Hz (hertz)), location sensing and orientation frequencies (5-10 kHz), pacing frequencies (0.5-5 kHz), or impedance frequencies (10-100 kHz). As a result, the amplitude of RF voltage seen at each of the RF outputs is nearly identical and equal to V_{RF} at the RF frequency only and allowed to differ for other purposes. FIG. 12 illustrates a way to use the multiple electrodes in connection with the RF frequency, so that they effectively act together as a single effective electrode and they are essentially isopotential at the RF. By keeping all the electrodes at almost the same voltage at any instance in time, they are electrically in parallel to effectively work as a single unit for RF even though they are not physically wired together. These active circuits allow for independent electrode use for electrogram sensing, location sensing and orientation, pacing, and impedance. In a further extension, the different electrodes may be driven slightly differently to achieve a more even distribution of delivered ablation energy while avoiding differences sufficient to lead to coagulation. Additional circuits monitor the applied voltage and individual currents and report the total power used as a conventional indicator. An alternative embodiment to the RF generator circuitry employs active circuit electronics to individually generate RF for each of the electrode elements 1-6. Clinical users directly choose the voltage (power) of one electrode and feedback electronics ensures the remaining electrodes obtain almost exactly that same voltage. This feedback is effective at frequencies close to the RF frequency of about 500 kHz (kilohertz). As a result, other frequencies are unaffected allowing simultaneous undisturbed use.

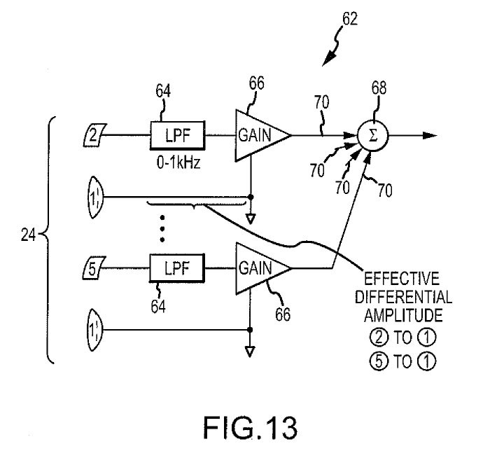

The catheter system further comprises electrogram sensing circuitry for sensing electrogram signals from the electrically active elements. FIG. 13 is a block diagram of electrogram sensing circuitry 62 with an LPF (low pass filter) 64 of between 0-1 kHz (kilohertz) and gain 66 from electrically active elements or electrodes 24. FIG. 13 illustrates an example of constructing a composite local bipolar electrogram signal 68 from individually generated electrogram signals 70. Electrogram signals and differential amplifiers may be used. Electrogram sensing circuitry exploits the ablation catheter's multiple electrodes and prevents interference of pacing, RF ablation, impedance sensing, and location sensing and orientation. With multiple electrodes, there is enhanced electrogram resolution. The electrogram sensing circuitry can sense unipolar or bipolar electrograms. For example, the electrogram sensing circuitry senses electrogram signals from the bipoles of the multiple closely spaced, small electrodes and creates the single composite electrogram signal to maximize detection of fractionated electrogram signals. Circuitry to sense electrogram voltages on the same catheter, as described above for RF ablation, operates at different frequencies (typically 0.1 Hz (hertz) to 1000 Hz) and may thus filter out RF energy and impedance signals.

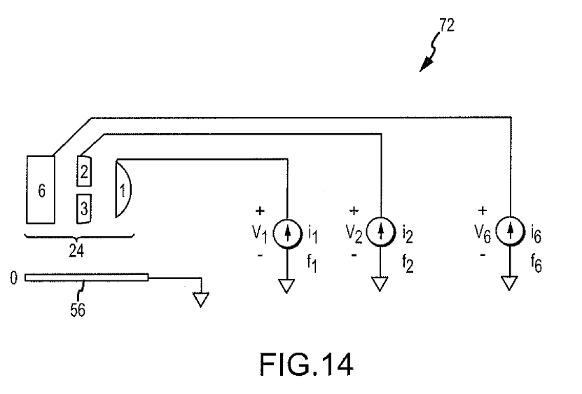

The catheter system further comprises impedance sensing circuitry comprised of catheter electrodes and one or more cutaneous distant return electrodes, as well as current sources (i) and voltage sensors (V). FIG. 14 is a block diagram of impedance sensing circuitry 72 for applying impedance current across the electrically active elements or electrodes 24 to the distant return electrode or electrically active element 56. Impedance sensing circuitry exploits the ablation catheter's additional electrodes and RF generator design to minimize interference with pacing, RF ablation, electrogram sensing, and location sensing and orientation. It is known that impedance is the ratio of voltage to current at a specific frequency. Additional circuits superimpose injected current of low amplitude and distinctive frequency to allow impedance measurement with or without concurrent ablation, and to better sense low amplitude, high frequency fractionated electrogram voltages across some combination of the multiple electrodes. Impedance sensor signal processing from the multi-electrode catheter provides a tissue proximity sensor as a result of impedance field alterations sensed in proximity to a cardiac surface. FIG. 14 is a diagram of impedance sensor implementation using the multiple electrodes as shown in FIG. 3 to dynamically choose the measurement most indicative of ablation induced tissue change. FIG. 14 shows voltages V₁, V₂, and V₆ across impedance sensor current sources i₁, i₂, and i₆, using band pass filters tuned to frequencies f₁, f₂, and f₆. One of the electrodes is designated as the best contact electrode based on the greatest change from the blood pool. Another one of the electrodes is designated as the next best contact electrode based on the next largest change from the blood pool, and so on. Impedance sensing currents are injected and voltages sensed on the catheter of the invention, but at different frequencies (typically 10 kHz (kilohertz) to 200 kHz) and filter out RF energy and electrogram signals. A 2-electrode impedance measurement is made using two distinct electrodes. A 3-electrode impedance measurement is made using three distinct electrodes. A preferred configuration includes at least one catheter electrode and one cutaneous distant electrode. The voltage sensing circuits are preferably connected across either adjacent catheter electrodes or a catheter electrode and a cutaneous distant electrode. A lesion assessment signal may be derived from two 3-electrode measurements using different frequencies but the same 3 electrodes. This 3-electrode measurement carries enhanced lesion assessment information and operates independently of RF ablation, electrogram sensing, and location sensing and orientation. Upon approaching tissue with the multi-electrode catheter, the sensed voltages change in a manner that is proximity and orientation dependent. Circuits process these impedance and other signals to enhance the accuracy of proximity measurements and enhance the accuracy of more conventional catheter-to-surface distances and cardiac anatomic locations.

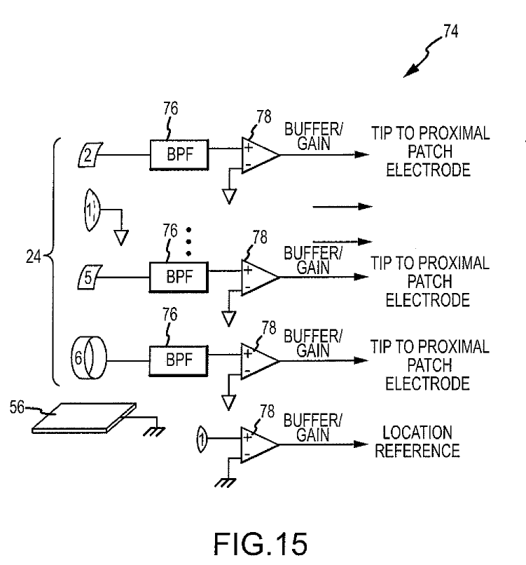

The catheter system further comprises location sensing and orientation circuitry for determining the catheter tip location and orientation. FIG. 15 is a block diagram of the location sensing and orientation circuitry 74. FIG. 15 shows BPF (band pass filter) 76 and buffer/gain 78 for the various multiple electrically active elements or electrodes 24, and shows distant return electrode 56 or proximal patch electrode. The location sensing and orientation circuitry exploits the ablation catheter's multiple electrodes and minimizes interference with pacing, RF ablation, electrogram sensing, and impedance sensing signals. Preferably, the location sensing and orientation circuitry is used with ENSITE NAVX, a three-dimensional navigation and visualization system comprising hardware and software obtained from St. Jude Medical of Minneapolis, Minn. (ENSITE NAVX is a registered trademark of St. Jude Medical, Atrial Fibrillation Division, Inc.). The catheter of the invention provides for an enhanced knowledge of the disposition of the tip electrodes using ENSITE NAVX because with the multiple electrodes, one can determine the orientation of the catheter tip in the ENSITE NAVX map. Further, the addition of multiple electrodes confers rotational information on the catheter. Knowledge of the rotational disposition with respect to a deflection angle that can occur when a pull wire is actuated may be used to provide information on the ENSITE NAVX system as to where deflection can occur when a pull wire is actuated. The pull wire may also be used to control the orientation of the catheter to increase or decrease contact. The electrically active elements or multiple electrodes provide full spatial localization, including three-dimensional positioning of the catheter and three-dimensional rotational orientation of the catheter. The electrically active elements or multiple electrodes provide an additional three (3) degrees of freedom of rotational orientation of the catheter in space, resulting in a total of six (6) degrees of freedom of positioning and orienting the catheter in space (i.e., 3 positioning degrees of freedom (x, y, z axes) and three additional rotational orientation degrees of freedom). FIG. 15 illustrates local differential sensing circuitry with the capacity to be turned back into standard unipolar location signals but with greater precision and less noise. In contrast, with conventional tip electrodes, the ENSITE NAVX information for such tip electrodes is only in terms of the x, y, and z coordinates of the centroid of the tip electrodes. With the multi-electrode ablation catheter of the invention, there is more information for the ENSITE NAVX system because there are more discrete electrodes to not just determine the location of the tip but also to determine its orientation in terms of its angle of attack with respect to where it is in space. This allows many benefits, including improved analysis of electrogram signals, improved knowledge regarding the necessary power delivery to the electrodes, and improved robotic control. For example, knowing the orientation of the catheter allows the system to adjust the power settings to provide transmural lesions at each active ablation electrode. The system (or the operator) can also use proximity information to adjust from a unipolar lesion (ablative current from the catheter electrode to a back patch) to a bipolar lesion (ablative current from one catheter electrode to another catheter electrode) or a combination/ratio of one to the other.

In addition, electrodes may be mounted on the catheter shaft (e.g., ring electrodes), and such electrodes may be spaced and sized to be compatible with RF ablation, as described above, in order to provide high quality location sensing and orientation and electrogram signals. The ENSITE NAVX location signals may be sensed by the same electrodes and specialized differential amplifiers to better determine catheter tip location and orientation. Local navigation field measurements are made by the collection of electrodes with substantially greater accuracy than possible for a single electrode. This accuracy results from the dynamic compensation of navigational field distortions that result from patch electrodes and anatomic conductivity variations. Closely spaced electrodes are localized with greater precision by using dedicated bipolar amplifiers and the catheter visualized or rendered using solid objects that correctly reflect the distribution of nontraditional, non-colinear multiple electrodes on a single catheter. These closely spaced electrodes also facilitate superior local characterization of the navigational field. As a result, greater navigational accuracy can be achieved to, for example, allow improved robotic catheter guidance from field-to-distance conversions.

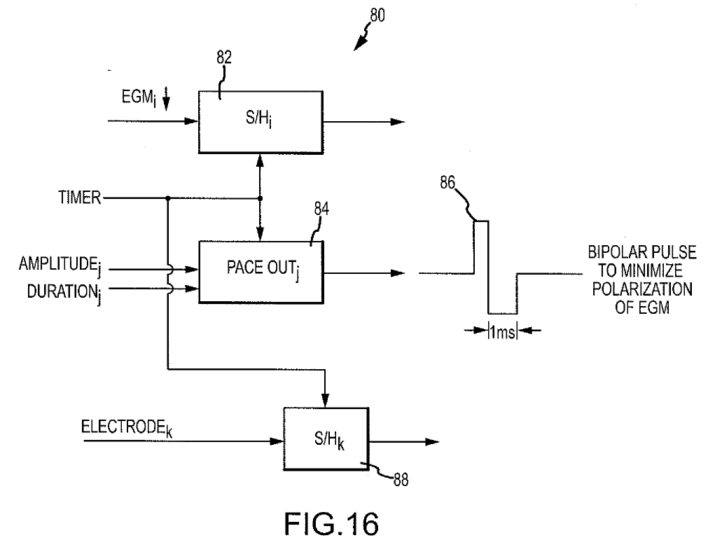

Optionally, the catheter system may further comprise pacing output circuitry for minimizing interference with impedance sensing, electrogram sensing, and location sensing and orientation. FIG. 16 is a block diagram of pacing output circuitry 80 to minimize impact on electrogram (EGM) sensing and S/Hᵢ (sample and hold) 82 and location sensing and orientation, to provide convenience of integrated pace stimulation or pace out (j) 84 and to bipolar pulse 86 to minimize polarization of electrogram sensing and to test for scar borders and determine ablation targets, and to provide electrode S/Hₖ (sample and hold). The pacing circuitry is integrated to the extent that the timing of the pace events is well-known and shared via a timer.

The invention further discloses a method to dynamically correct for navigational field inhomogenicties and to dynamically compensate for navigational field distortions in blood that result from anatomical current concentration in conductive blood vessels and other variations of tissue conductivity and location sensing and orientation field electrodes. This compensation permits improved map generation and navigational accuracy as well as provides better correspondence with images obtained by other means such as MRI (Magnetic Resonance Imaging). A collection of closely spaced electrodes (indexed by i=1, . . . , N) on a rigid body was studied. Catheter deformation in the vicinity of these N electrodes was presumed negligible allowing the rigid body approximation. For the ENSITE NAVX, navigational fields were applied in a variety of directions which was simplified in this treatment to consist of three directions and index them j=x, y, and z. The rigid body's center position and orientation in space was represented by a 6×1 vector (of displacements and orientation angles) denoted x. The collection of observed voltages vⱼⁱ was combined into a 3N×1 vector, v. Although most generally these voltages were functions of both x and time t, cardiac motions and the effects of ventilation were assumed to have been filtered out or otherwise compensated for. As a result, the vector equation was written v=f(x). Using the chain rule, the time derivatives of these voltages were the following: \dot{v}=Dₓf(x)·\dot{x}=J(x)·\dot{x}, where Dₓ is the derivative operator denoting differentiation with respect to each element of x of each function that defines a voltage. The resulting 3N×6 matrix of partial derivatives is commonly known as a Jacobian matrix which was denoted by J. The Jacobian's elements may be determined, for example, by empiric calibration methods with least squares fits for a particular multi-electrode catheter design. If one could be assured the rigid body would only translate and never change its orientation, x collapses to a 3×1 displacement vector and the rows of J are simply the gradients of each scalar potential field for each voltage vⱼⁱ in v. The elements of J were then recognizable as local electric field components that would exist at the center of the rigid body (had it not been there) which was determined from a collection of closely spaced electrodes. As noted above for the more general case, J was a 3N×6 matrix that was determined for a particular catheter and multi-electrode combination by some combination of empiric calibration or analytic solution. The compensated location and orientation of this rigid body was solved by inverting the Jacobian matrix and thus

\dot{x}=J⁻¹(x)·\dot{v}

x(t)=∫J⁻¹(x(t))·\dot{v}(t)dt.

In the case where N>2, the situation was overdetermined and a generalized (least squares) inverse to most harmoniously determine the catheter's position and orientation, now dynamically compensated for navigation field inhomogenieties was used. Non-contact cardiac mapping of electrical activity was also obtained from the inventive catheter's multiple electrodes. Using mathematics based on balloon array non-contact mapping, which explicitly accounts for the normal current to the non-conductor being equal to zero, local mapping of endocardial potentials was performed without requiring electrode contact. The use of a small and flexible catheter region, compared to the large balloon array, constituted an advantage-allowing one to effectively “zoom in” for better detail. Instead of the “entire chamber at once” of the balloon array or the “one point at a time” approach of ENSITE Diagnostic Landmark (D×L) maps, the catheter of the invention constituted a hybrid approach. A superior monophasic action potential like signal from this mapping or ablation catheter is further provided if the electrodes protrude slightly from the catheter tip (see FIG. 4) and thereby concentrate contact force and local deformation.

Although a number of representative embodiments according to the teachings have been described above with a certain degree of particularity, those skilled in the art could make numerous alterations to the disclosed embodiments without departing from the scope of this invention. For example, different types of catheters may be manufactured or result from the inventive process described in detail above. For instance, catheters used for diagnostic purposes and catheters used for therapeutic purposes may both be manufactured using the inventive process. Additionally, all directional references (e.g., upper, lower, upward, downward, left, right, leftward, rightward, top, bottom, above, below, vertical, horizontal, clockwise, and counterclockwise) are only used for identification purposes to aid the reader's understanding of the invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the invention as defined in the appended claims.

What is claimed is:

1. A catheter comprising:
an elongated catheter shaft having a proximal end, a distal end, and a lumen therethrough; and,
a catheter tip at the distal end of the catheter shaft having two or more closely spaced electrically active elements mounted on the catheter tip, wherein the electrically active elements are coupled to a plurality of electronic circuitries used for electrogram sensing, impedance sensing, and location sensing and orientation.

2. The catheter of claim 1 wherein the electrically active elements are selected from the group consisting of ablation electrodes, electrogram sensing electrodes, contact sensing electrodes, pacing electrodes, location electrodes, tissue impedance sensors, and electrical sensors.

3. The catheter of claim 1 wherein the electrically active elements are spaced apart from each other a distance of 0.1 millimeter to 0.3 millimeter.

4. The catheter of claim 1 wherein the electrically active elements are positioned circumferentially around the catheter tip.

5. The catheter of claim 1 wherein the electrically active elements each have one or more protrusions on an outer surface.

6. The catheter of claim 1 wherein the catheter tip has six electrically active elements mounted on the tip.

7. The catheter of claim 1 wherein the plurality of electronic circuitries comprises an electrogram sensing circuitry, an impedance sensing circuitry, a location sensing and orientation circuitry, and an RF generator circuitry.

8. The catheter of claim 7 wherein the plurality of electronic circuitries further comprises a pacing output circuitry.

9. The catheter of claim 1 wherein the catheter is an RF ablation sensing catheter.

10. The catheter of claim 1 wherein the catheter is assembled in a piece part assembly by mating the catheter shaft which is pre-manufactured with the catheter tip which is pre-manufactured.

11. The catheter of claim 1 wherein the electrically active elements provide three-dimensional positioning of the catheter and three-dimensional rotational orientation of the catheter.

12. A catheter system comprising:
a catheter having an elongated catheter shaft with a proximal end, a distal end, and a lumen therethrough, and having a catheter tip at the distal end of the catheter shaft with a plurality of closely spaced electrically active elements mounted on the catheter tip;
RF generator circuitry for applying RF energy across the electrically active elements to a distant return electrically active element;
electrogram sensing circuitry for sensing electrogram signals from the electrically active elements;
impedance sensing circuitry for applying impedance current across the electrically active elements to the distant return electrically active element; and,
location sensing and orientation circuitry for determining the catheter tip location and orientation.

13. The catheter system of claim 12 further comprising pacing output circuitry for minimizing interference with impedance sensing and location sensing and orientation.

14. The catheter system of claim 12 wherein the electrically active elements are selected from the group consisting of ablation electrodes, electrogram sensing electrodes, contact sensing electrodes, pacing electrodes, location electrodes, tissue impedance sensors, and electrical sensors.

15. The catheter system of claim 12 wherein the electrically active elements are spaced apart from each other a distance of 0.1 millimeter to 0.3 millimeter.

16. The catheter system of claim 12 wherein the catheter system corrects for navigational field inhomogenieties and compensates for navigational field distortions in blood.

17. The catheter system of claim 12 wherein the electrically active elements each have one or more protrusions on an outer surface.

18. An ablation sensing catheter system comprising:
a catheter having an elongated catheter shaft with a proximal end, a distal end, and a lumen therethrough, and having a catheter tip at the distal end of the catheter shaft with a plurality of closely spaced electrodes mounted on the catheter tip;
RF generator circuitry for applying RF energy across the electrodes to a distant return electrode;
electrogram sensing circuitry for sensing electrogram signals from the electrodes;
impedance sensing circuitry for applying impedance current across the electrodes to the distant return electrode; and,
location sensing and orientation circuitry for determining the catheter tip location and orientation.

19. The catheter system of claim 18 further comprising pacing output circuitry for minimizing interference with impedance sensing and location sensing and orientation.

20. The catheter system of claim 18 wherein the electrodes are spaced apart from each other a distance of 0.1 millimeter to 0.3 millimeter.

21. The catheter system of claim 18 wherein the catheter system corrects for navigational field inhomogenieties and compensates for navigational field distortions in blood.
